(19) United States
(12) Patent Application Publication
(10) Pub. No.: US 2024/0180455 A1
(43) Pub. Date: Jun. 6, 2024

(54) TRANSCUTANEOUS MUSCLE OXYGEN SATURATION DETECTION DEVICE

(71) Applicant: Japan Display Inc., Tokyo (JP)

(72) Inventors: Yasushi TOMIOKA, Tokyo (JP); Akio TAKIMOTO, Tokyo (JP); Hirofumi KATO, Tokyo (JP); Takashi NAKAMURA, Tokyo (JP); Chiharu KABURAGI, Tokyo (JP); Hiroyuki KIMURA, Tokyo (JP); Miharu MATSUSHIMA, Tokyo (JP)

(73) Assignee: Japan Display Inc., Tokyo (JP)

(21) Appl. No.: 18/437,293

(22) Filed: Feb. 9, 2024

Related U.S. Application Data
(63) Continuation of application No. PCT/JP2022/025322, filed on Jun. 24, 2022.

(30) Foreign Application Priority Data
Aug. 10, 2021 (JP) 2021-130855

(51) Int. Cl. A61B 5/1455

(52) U.S. Cl. CPC A61B 5/14552; A61B 2562/0238; A61B 2562/0242

(57) ABSTRACT

According to an aspect, a transcutaneous muscle oxygen saturation detection device includes: a light source configured to emit light into a body; and a first light detector and a second light detector that are configured to detect reflected light reflected in the body. The second light detector is circumferentially displaced about the light source from a first imaginary line connecting the light source to the first light detector.

### CROSS-REFERENCE TO RELATED APPLICATION

This application claims the benefit of priority from Japanese Patent Application No. 2021-130855 filed on Aug. 10, 2021 and International Patent Application No. PCT/JP2022/025322 filed on Jun. 24, 2022, the entire contents of which are incorporated herein by reference.

### BACKGROUND

### 1. Technical Field

What is disclosed herein relates to a transcutaneous muscle oxygen saturation detection device.

### 2. Description of the Related Art

An oxygen saturation level in blood (hereinafter referred to as a “blood oxygen saturation level (SpO₂)”) refers to the ratio of the amount of oxygen actually bound to hemoglobin to the total amount of oxygen under the assumption that the oxygen is bound to all the hemoglobin in the blood. Methods for detecting such a blood oxygen saturation level (SpO₂) include, first, a method of collecting blood from an artery and measuring the amount of oxygen. Secondly, there is a method in which a detection device is used to emit light into a body through the skin, and detect the blood oxygen saturation level (SpO₂) based on light transmitted through or reflected from the artery. Since the second detection method is performed through the skin, the detection device may be referred to as a transcutaneous oxygen saturation detection device. A transcutaneous oxygen saturation detection device of U.S. Pat. No. 8,941,830 (U.S. Pat. No. 8,941,830) includes one light source that emits light into the body and two light detectors that receive reflected light reflected from inside the body.

In recent years, detection of oxygen saturation levels in muscle tissues (hereinafter referred to as “muscle oxygen saturation levels (SmO₂)”) of athletes has been conducted. The detection method is such that, similarly to the detection of the blood oxygen saturation level (SpO₂), light is emitted into the body through the skin, and the muscle oxygen saturation level (SmO₂) is detected based on light transmitted through or reflected from capillaries in muscle tissues. However, if the light transmitted through or reflected from the capillaries in the muscle tissues further penetrates the arteries, the light contains information on the blood oxygen saturation level (SpO₂) of the artery. Therefore, the muscle oxygen saturation level (SmO₂) cannot be detected accurately.

In the transcutaneous oxygen saturation detection device of U.S. Pat. No. 8,941,830, the one light source and the two light detectors are arranged in a straight line. With such an arrangement, if the one light source and one of the light detectors overlap an artery, the other of the light detectors may also overlap the artery. That is, although two light detectors are provided, the muscle oxygen saturation level (SmO₂) may fail to be accurately obtained. If the muscle oxygen saturation level (SmO₂) cannot be accurately obtained, a need for a burdensome operation arises in which the transcutaneous oxygen saturation detection device is reattached to another location and the detection is performed again. Because of the above problem, development is desired for a highly convenient transcutaneous muscle oxygen saturation detection device that can avoid the burdensome operation (reattachment).

For the foregoing reasons, there is a need for a highly convenient transcutaneous muscle oxygen saturation detection device.

### SUMMARY

According to an aspect, a transcutaneous muscle oxygen saturation detection device includes: a light source configured to emit light into a body; and a first light detector and a second light detector that are configured to detect reflected light reflected in the body. The second light detector is circumferentially displaced about the light source from a first imaginary line connecting the light source to the first light detector.

### BRIEF DESCRIPTION OF THE DRAWINGS

FIG. 1 is a plan view schematically illustrating a transcutaneous muscle oxygen saturation detection device according to a first embodiment of the present disclosure;

FIG. 2 is a diagram illustrating paths of light when the light has entered the body of a subject of examination;

FIG. 3 is a diagram illustrating absorption coefficients of red light and infrared light;

FIG. 4 is a view illustrating a state in which the transcutaneous muscle oxygen saturation detection device is attached to the right leg of the subject of examination;

FIG. 5 is a plan view schematically illustrating a transcutaneous muscle oxygen saturation detection device according to a second embodiment of the present disclosure;

FIG. 6 is a plan view schematically illustrating a transcutaneous muscle oxygen saturation detection device according to a third embodiment of the present disclosure;

FIG. 7 is a sectional view as viewed along arrows VII-VII in FIG. 6;

FIG. 8 is a plan view schematically illustrating a transcutaneous muscle oxygen saturation detection device according to a fourth embodiment of the present disclosure;

FIG. 9 is a sectional view as viewed along arrows IX-IX in FIG. 8;

FIG. 10 is a plan view schematically illustrating a transcutaneous muscle oxygen saturation detection device according to a fifth embodiment of the present disclosure;

FIG. 11 is a plan view schematically illustrating a transcutaneous muscle oxygen saturation detection device according to a sixth embodiment of the present disclosure;

FIG. 12 is a plan view schematically illustrating a transcutaneous muscle oxygen saturation detection device according to a seventh embodiment of the present disclosure;

FIG. 13 is a plan view schematically illustrating a transcutaneous muscle oxygen saturation detection device according to an eighth embodiment of the present disclosure, further illustrating areas of light detection devices that are driven at first to third lighting times; and

FIG. 14 is a plan view illustrating areas of the light detection devices that are driven at fourth to sixth lighting times in the transcutaneous muscle oxygen saturation detection device of the eighth embodiment.

### DETAILED DESCRIPTION

The following describes modes (embodiments) for carrying out the present disclosure in detail with reference to the drawings. The present disclosure is not limited to the description of the embodiments given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the invention. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the description and the drawings, and detailed description thereof may be omitted as appropriate.

In the present specification and claims, in expressing an aspect of disposing another structure on or above a certain structure, a case of simply expressing “on” includes both a case of disposing the other structure immediately on the certain structure so as to contact the certain structure and a case of disposing the other structure above the certain structure with still another structure interposed therebetween, unless otherwise specified.

### First Embodiment

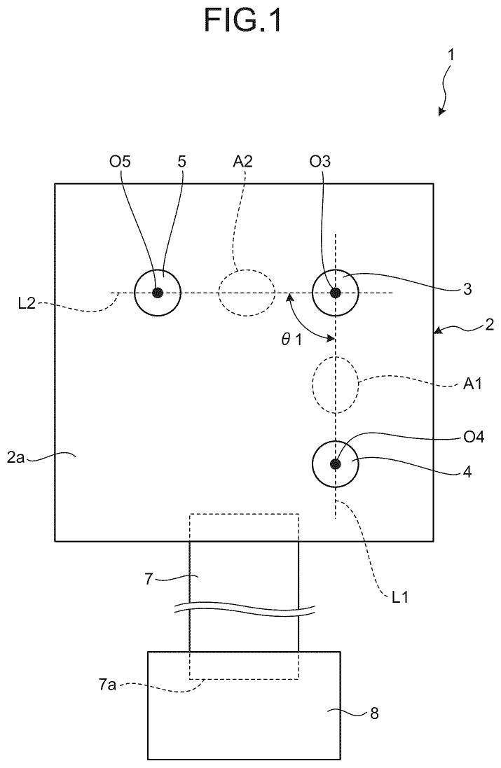

FIG. 1 is a plan view schematically illustrating a transcutaneous muscle oxygen saturation detection device according to a first embodiment of the present disclosure. In the first embodiment, a transcutaneous muscle oxygen saturation detection device 1 having a basic configuration will be described. As illustrated in FIG. 1, the transcutaneous muscle oxygen saturation detection device 1 of the first embodiment includes a sheet 2, one light source 3, and two light detectors (first light detector 4 and second light detector 5). Hereinafter, a direction parallel to a line normal to the sheet 2 is referred to as an “orthogonal direction”. In addition, the term “plan view” hereinafter refers to viewing in the orthogonal direction.

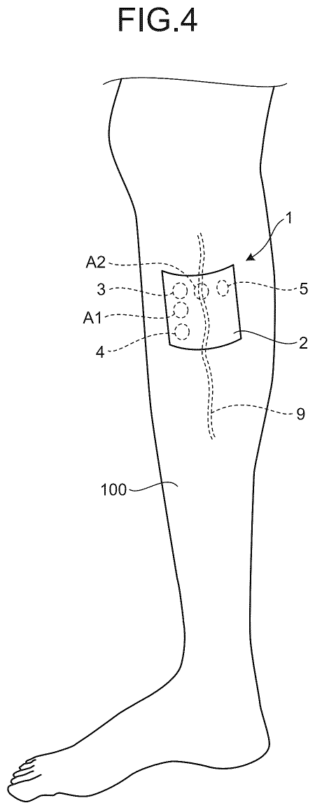

The sheet 2 is a support plate that supports the light source 3 and the light detectors (first light detector 4 and second light detector 5). The sheet 2 is formed in a quadrilateral shape in plan view. A surface 2a of the sheet 2 is a surface to be directed and attached to an arm or a leg of a subject of examination (refer to FIG. 4). The light source 3, the first light detector 4, and the second light detector 5 are mounted on the surface 2a.

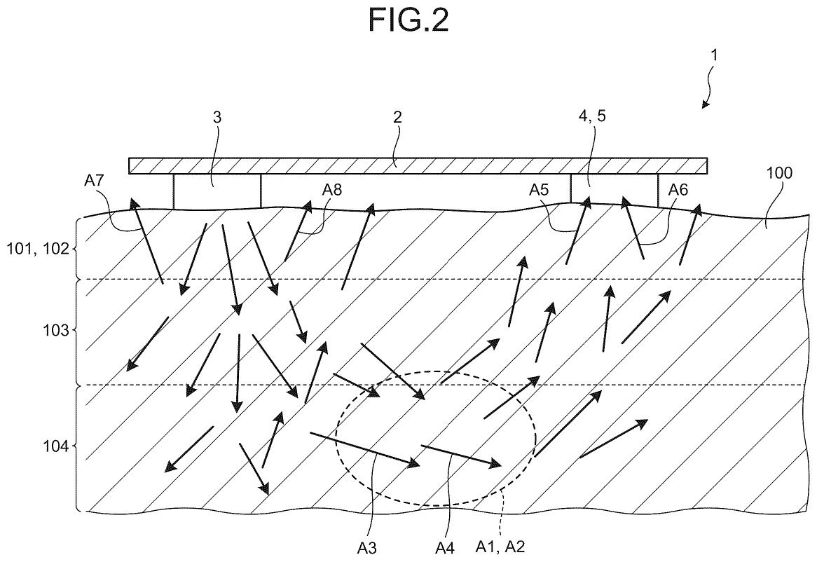

The sheet 2 is made of a flexible material. Therefore, the sheet 2, when attached to the subject of examination, deforms into a shape along the outer surface of the arm or the leg. As a result, the light source 3, the first light detector 4, and the second light detector 5 fixed to the surface 2a of the sheet 2 come in contact with a body 100 (refer to FIG. 2).

A flexible printed circuit board 7 is provided at one side of the sheet 2. A terminal 7a is provided at an end of the flexible printed circuit board 7. The flexible printed circuit board 7 is fixed to the sheet 2 such that the terminal 7a protrudes from the one side of the sheet 2 to the outside of the sheet 2. The sheet 2 is provided with various types of wiring. One end of each of the various types of wiring is coupled to one of the light source 3, the first light detector 4, and the second light detector 5, and the other end extends to the terminal 7a of the flexible printed circuit board 7. The terminal 7a of the flexible printed circuit board 7 is inserted into a connector of a control device 8. With this configuration, the light source 3, the first light detector 4, and the second light detector 5 receive various signals from the control device 8 or transmits signals (detection results) to the control device 8.

The light source 3 is fixed to the sheet 2 so as to emit light in the direction orthogonal to the surface 2a of the sheet 2. The light source 3 emits light rays having two wavelengths. One of the light rays having the two wavelengths is light having a wavelength from 600 nm to less than 800 nm. The light having a wavelength from 600 nm to less than 800 nm is red visible light, and may be hereinafter referred to as “red light” or “R”. The other of the light rays having the two wavelengths is light having a wavelength from 800 nm to less than 1000 nm. The light having a wavelength from 800 nm to less than 1000 nm is infrared light, and may be hereinafter referred to as “infrared light” or “IR”.

The light source 3 of the present embodiment includes a first light-emitting element that emits the red light and a second light-emitting element that emits the infrared light, which are not illustrated. In the present embodiment, light-emitting diodes (LEDs) are used as the first light-emitting element and the second light-emitting element. The first light-emitting element of the embodiment emits the red light mainly having a wavelength of 665 nm. The second light-emitting element emits the infrared light mainly having a wavelength of 880 nm. The first and the second light-emitting elements are alternately turned on in a time-division manner, so that the light rays having the two wavelengths are alternately emitted from the light source 3.

The light source of the present disclosure only needs to be capable of emitting the light rays having the two wavelengths of the red light and the infrared light, and need not include two light-emitting elements. The oxygen saturation detection device of the present disclosure is not limited to one that alternately emits the red light (R) and the infrared light (IR) in a time-divisional manner, but may be one that sequentially or simultaneously emits the red light and the infrared light.

FIG. 2 is a diagram illustrating paths of light when the light has entered the body of the subject of examination. As illustrated in FIG. 2, when detecting the muscle oxygen saturation level (SmO₂), the light source 3 emits the light toward epidermis 101 of the body 100. As a result, the light enters the body 100.

Tissues of the body 100 include: the epidermis 101, dermis 102, a subcutaneous tissue 103, and a muscle tissue 104 arranged in this order from the outside. FIG. 2 illustrates the epidermis 101 and the dermis 102 in an integrated manner. Blood (in capillaries) flows through the dermis 102, the subcutaneous tissue 103, and the muscle tissue 104. Hereinafter, the direction in which the muscle tissue 104 is located as viewed from the epidermis 101 is referred to as a “depth direction”.

The light emitted from the light source 3 passes through the epidermis 101 and penetrates in the depth direction. The light is reflected at any part of the body 100 in the process of the penetration and is emitted out of the body 100 as reflected light. Of the reflected light, the light (reflected light) that has reached the muscle tissue 104 and passed through the blood flowing through the muscle tissue 104, contains information on the muscle oxygen saturation level (SmO₂). In contrast, the light (reflected light) that has only reached the subcutaneous tissue 103 and passed through the blood flowing through the subcutaneous tissue 103, does not contain the information on the muscle oxygen saturation level (SmO₂).

In addition, the light attenuates in the process of passing through various parts of the body 100. Therefore, to ensure intensity of the light, the light source 3 preferably has a narrow orientation range, in other words, high directivity of light. In the body 100, the transmittance of the infrared light (IR) is higher than that of the red light (R). Therefore, the intensity of red light (R) is preferably higher than that of the infrared light (IR). The following describes the light detectors.

As illustrated in FIG. 1, the first and the second light detectors 4 and 5 are each a photodiode. A back surface opposite a light-receiving surface of each of the first and the second light detectors 4 and 5 is fixed to the surface 2a of the sheet 2. That is, the light-receiving surface of each the first and the second light detectors 4 and 5 faces in the same direction as the surface 2a of the sheet 2. Each of the first and the second light detectors 4 and 5 receives the reflected light emitted out of the body 100. The first and the second light detectors 4 and 5 transmit, to the control device 8, electrical signals each corresponding to an amount of light received. The reflected light received by the first and the second light detectors 4 and 5 includes both the red light emitted from the first light-emitting element and the infrared light emitted from the second light-emitting element.

The first and the second light detectors 4 and 5 are each at a distance of approximately 10 mm or more from the light source 3. The reason for this will be described with reference to FIG. 2. Arrows in FIG. 2 indicate light penetrating in the body 100. This is because, as illustrated in FIG. 2, light changes its direction of travel in the process of the penetration in the interior of the body 100. That is, when the light path in the body 100 is long (when the light reaches the muscle tissue 104 located at a deep portion of the body 100 as illustrated by arrows A3 and A4 in FIG. 2), the distance away from the light source 3 in a radial direction (direction away from the light source 3) also increases (refer to arrows A5 and A6 in FIG. 2). Therefore, the light (reflected light) that has reached the muscle tissue 104 is dispersed to locations near or far from the light source 3 and emitted out of the body 100. In contrast, when the light path in the body 100 is short (when, for example, the light is reflected at the dermis 102 as illustrated by arrows A7 and A8 in FIG. 2), the distance away from the light source 3 is relatively shorter. That is, the light is emitted out of the body 100 near the light source 3. In view of the above, the first and the second light detectors 4 and 5 are located at distances where the reflected light that has not reached the muscle tissue 104 cannot be received while the reflected light that has reached the muscle tissue 104 can be received.

Therefore, the first light detector 4 receives a relatively large amount of light (including both the R and the IR) that has passed through a portion of the muscle tissue 104 located between the light source 3 and the first light detector 4 (refer to an area surrounded by a dashed line A1 in FIGS. 1 and 2). The second light detector 5 receives a relatively large amount of light (including both the R and the IR) that has passed through a portion of the muscle tissue 104 located between the light source 3 and the second light detector 5 (refer to an area surrounded by a dashed line A2 in FIGS. 1 and 2).

As illustrated in FIG. 1, the first and the second light detectors 4 and 5 of the first embodiment are at the same distance from the light source 3. The first and the second light detectors 4 and 5 are located in positions that form an angle of 90 degrees about the light source 3 in plan view. In other words, a first imaginary line L1 intersects a second imaginary line L2 with the center O3 of the light source 3 serving as the intersection point. The first imaginary line L1 is an imaginary line connecting a center O3 of the light source 3 to a center O4 of the first light detector 4, and the second imaginary line L2 is an imaginary line connecting the center O3 of the light source 3 to a center O5 of the second light detector 5. The first imaginary line L1 intersects the second imaginary line L2 at an angle of 90 degrees at the intersection point. As described above, the second light detector 5 is circumferentially displaced about the light source 3 from the first imaginary line L1 connecting the light source 3 to the first light detector 4. For clarity, the first and the second imaginary lines L1 and L2 in FIG. 1 are illustrated so as to extend beyond the centers O3, O4, and O5.

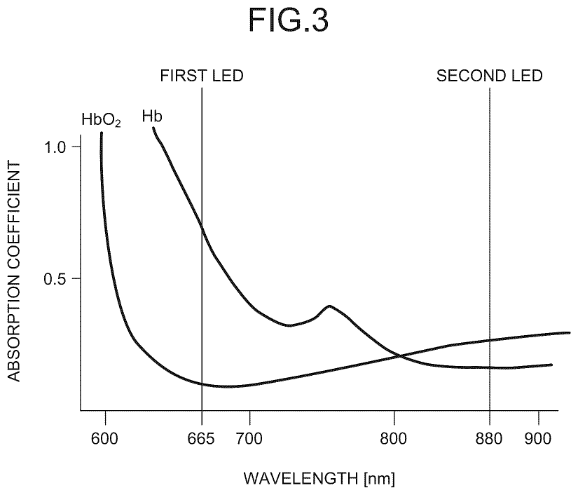

The control device 8 calculates the oxygen saturation level based on the amount of light received that is indicated by the electrical signal transmitted from each of the first and the second light detectors 4 and 5. The following briefly describes a method for calculating the oxygen saturation level with reference to FIG. 3.

FIG. 3 is a diagram illustrating absorption coefficients of the red light and the infrared light. The absorption coefficient of light on the vertical axis in FIG. 3 has a property that light is more easily absorbed as the value is larger. Hb in FIG. 3 is hemoglobin not bound to oxygen. HbO₂ in FIG. 3 is hemoglobin bound to oxygen.

Red blood cells contained in blood contain hemoglobin. Hemoglobin has a dark red color when not bound to oxygen, and has a bright red color when bound to oxygen. Therefore, the absorption coefficient of the red light differs between the hemoglobin bound to oxygen (HbO₂) and the hemoglobin not bound to oxygen (Hb). In detail, as illustrated in FIG. 3, the hemoglobin not bound to oxygen (Hb) has a higher absorption coefficient of the red light (R) than that of the hemoglobin bound to oxygen (HbO₂). Therefore, when the red light (R) passes through the blood, more red light is transmitted (reflected) as the hemoglobin bound to oxygen (HbO₂) increases. In contrast, less red light is transmitted (reflected) as the hemoglobin not bound to oxygen (Hb) increases. Thus, the amount of the hemoglobin bound to oxygen (HbO₂) can be relatively determined based on the amount of reflected light of the first light-emitting element (red light having a wavelength of 665 nm) received by each of the first and the second light detectors 4 and 5.

In contrast, as illustrated in FIG. 3, the absorption coefficient of infrared light (IR) does not differ much between the hemoglobin not bound to oxygen (Hb) and the hemoglobin bound to oxygen (HbO₂). That is, the infrared light (IR) decreases in proportion to the overall amount of hemoglobin penetrated thereby. Therefore, the overall amount of hemoglobin can be determined based on the amount of reflected light of the second light-emitting element (infrared light having a wavelength of 880 nm) received by each of the first and the second light detectors 4 and 5. Then, the muscle oxygen saturation level (SmO₂) is calculated by obtaining a ratio (R/IR) of the amount of red light (R) that has been received to the amount of infrared light (IR) that has been received.

The detection of the muscle oxygen saturation level (SmO₂) by the control device 8 is performed for each of the light detectors. The transcutaneous muscle oxygen saturation detection device 1 of the first embodiment includes the two light detectors: the first light detector 4 and the second light detector 5. Thus, the control device 8 calculates the muscle oxygen saturation level (SmO₂) of the portion of the muscle tissue 104 located between the light source 3 and the first light detector 4 (refer to the area surrounded by the dashed line A1 in FIGS. 1 and 2) based on the amount of the red light (R) received and the amount of the infrared light (IR) received that are indicated by the electrical signals transmitted from the first light detector 4. In the same manner, the control device 8 calculates the muscle oxygen saturation level (SmO₂) of the portion of the muscle tissue located between the light source 3 and the second light detector 5 (refer to the area surrounded by the dashed line A2 in FIGS. 1 and 2) based on the amount of the red light (R) received and the amount of the infrared light (IR) received that are indicated by the electrical signals transmitted from the second light detector 5.

Based on the detection results of the first and the second light detectors 4 and 5, the control device 8 determines whether information on the oxygen saturation level of an artery is included. The artery pulsates, and the amount of the hemoglobin bound to oxygen (HbO₂) and the total amount of hemoglobin change in a short time. Therefore, when light passes through the artery, the amount of the red light (R) and the amount of the infrared light (IR) received by the light detectors also change over time. Therefore, when the amount of received light detected by the light detectors has changed in a short time, the control device 8 determines that the accuracy of the detected muscle oxygen saturation level (SmO₂) is low because the information on the blood oxygen saturation level (SpO₂) of the artery is included.

FIG. 4 is a view illustrating a state in which the transcutaneous muscle oxygen saturation detection device is attached to the right leg of the subject of examination. The following describes a method for attaching the transcutaneous muscle oxygen saturation detection device 1 of the first embodiment. As illustrated in FIG. 4, the transcutaneous muscle oxygen saturation detection device 1 is attached to a portion (in the present embodiment, the calf of the right leg) where the muscle oxygen saturation level (SmO₂) is to be detected with the surface 2a of the sheet 2 facing the portion. The sheet 2 is then fixed with a supporter or a stretching band, which are not illustrated, to prevent the sheet 2 from being displaced.

Regarding the orientation of the transcutaneous muscle oxygen saturation detection device 1, the direction of arrangement of the light source 3 and the first light detector 4 (extending direction of the first imaginary line L1) is aligned to be parallel to the longitudinal direction of a measurement site (in the case of an arm, the extending direction of the arm, or in the case of a leg, the extending direction of the leg). The artery extends in the longitudinal direction. With this orientation, the first and the second light detectors 4 and 5 are arranged so as not to be lined up in the longitudinal direction of the measurement site. In other words, the respective detected portions (areas surrounded by the dashed lines A1 and A2) do not both overlap the artery. Therefore, as illustrated in FIG. 4, even if an artery 9 overlaps a gap between the light source 3 and the second light detector 5 (area surrounded by the dashed line A2), the artery 9 does not overlap a gap between the light source 3 and the first light detector 4 (area surrounded by the dashed line A1). Therefore, the accurate muscle oxygen saturation level (SmO₂) can be detected with a high probability from the detection results of the first light detector 4.

As described above, with the transcutaneous muscle oxygen saturation detection device 1 of the first embodiment, the muscle oxygen saturation level (SmO₂) is detected with a high probability. Therefore, a burdensome operation of reattaching the sheet 2 and performing the detection again can be avoided, thus increasing the convenience.

In the first embodiment, the first imaginary line L1 intersects the second imaginary line L2 at an angle of 90 degrees at the intersection point, but the present disclosure is not limited to this embodiment. The angle only needs to prevent the first and the second light detectors 4 and 5 from being lined up in the longitudinal direction of the measurement site, that is, the crossing angle only needs to be 5 degrees or larger. The following describes other embodiments of the present disclosure. In the following description, the same components as those described in the first embodiment are denoted by the same reference numerals, and the description thereof will be omitted.

### Second Embodiment

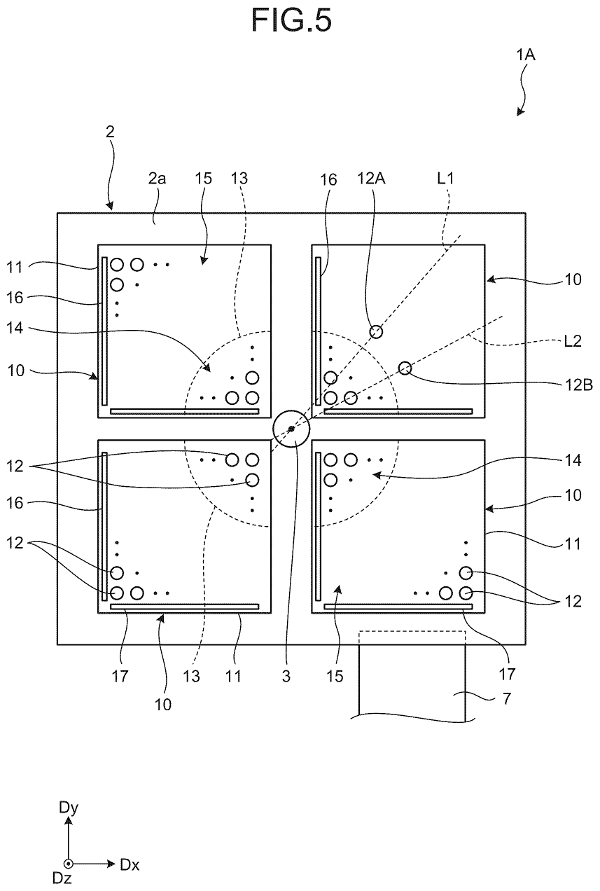

FIG. 5 is a plan view schematically illustrating a transcutaneous muscle oxygen saturation detection device according to a second embodiment of the present disclosure. As illustrated in FIG. 5, a transcutaneous muscle oxygen saturation detection device 1A of the second embodiment differs from the transcutaneous muscle oxygen saturation detection device 1 of the first embodiment in terms of including four light detection devices 10. The following description focuses on the differences.

Each of the light detection devices 10 includes a substrate 11 and a plurality of light detectors 12 provided on the substrate 11. The substrate 11 includes thin-film transistors (TFTs), such as switching elements and various types of wiring, and is called a backplane or an array substrate.

When the light detection device 10 is viewed in plan view, the rim portion of the light detection device 10 that forms a frame shape serves as a non-detection area. The non-detection area is provided with a scan line drive circuit 16 and a signal line processing circuit 17. An area surrounded by the non-detection area serves as a detection area where the light detectors 12 are arranged. The light detectors 12 are arranged in a matrix having a row-column configuration in the detection area and arranged in a first direction Dx and a second direction Dy.

The first direction Dx mentioned above is a direction parallel to the substrate 11. The second direction Dy is a direction parallel to the substrate 11 and is a direction intersecting the first direction Dx. In the present embodiment, the second direction Dy is orthogonal to the first direction Dx. A direction orthogonal to each of the first direction Dx and the second direction Dy is called a third direction Dz. A case of viewing from the third direction Dz is referred to as “plan view” in the same manner as in the first embodiment.

The scan line drive circuit 16 is a circuit that drives a plurality of scan lines based on various control signals from the control device 8 (refer to FIG. 1). The scan line drive circuit 16 sequentially or simultaneously selects the scan lines and supplies drive signals to the selected scan lines. The signal line processing circuit 17 is a circuit that sequentially or simultaneously selects a plurality of output signal lines and couples the selected signal lines to the control device 8 (refer to FIG. 1). The signal line processing circuit 17 converts analog signals to be transmitted to the control device 8 (refer to FIG. 1) through the output signal line into digital signals. Thus, the detection results of the light detectors 12 provided in each of the four light detection devices 10 are transmitted to the control device 8.

The four light detection devices 10 are fixed to the sheet 2 so as to form two rows in each of the first direction Dx and the second direction Dy. The light source 3 is fixed to the sheet 2 so as to be located at a central portion of the four light detection devices 10.

A dashed line 13 in FIG. 5 is the boundary line where the distance from the light source 3 is approximately 10 mm. In each of the four light detection devices 10, some of the light detectors 12 are arranged in a near area 14 within approximately 10 mm from the light source 3. The rest of light detectors 12 are arranged in a far area 15 at a distance of approximately 10 mm or more from the light source 3. Light detectors 12A and 12B located in the far area 15 correspond to the first and the second light detectors 4 and 5 described in the first embodiment. That is, the first imaginary line L1 intersects the second imaginary line L2 with the center O3 of the light source 3 serving as the intersection point. The first imaginary line L1 is an imaginary line passing through the light source 3 and the light detector 12A, and the second imaginary line L2 is an imaginary line passing through the light source 3 and the light detector 12B. Thus, the light detectors 12 include the first and the second light detectors 4 and 5.

With the transcutaneous muscle oxygen saturation detection device 1A of the second embodiment described above, the reflected light is received by each of the light detectors 12 arranged in the first direction Dx and the second direction Dy. Therefore, the muscle oxygen saturation level (SmO₂) can be detected for each area obtained by dividing the muscle tissue 104 in the first direction Dx and the second direction Dy. Thus, the detailed muscle oxygen saturation level (SmO₂) can be obtained.

The transcutaneous muscle oxygen saturation detection device 1A of the second embodiment includes the four light detection devices 10 and has a larger detection area than that of the transcutaneous muscle oxygen saturation detection device 1 of the first embodiment. That is, the probability of detecting the accurate muscle oxygen saturation level (SmO₂) without overlapping the artery 9 is very high. Therefore, the burdensome operation of reattaching the sheet 2 and performing the detection again can be avoided, thus increasing the convenience.

The reflected light received by the light detectors 12 arranged in the far area 15 has passed through the dermis 102 and the subcutaneous tissue 103 while traveling from the epidermis 101 until reaching the muscle tissue 104 and traveling from the muscle tissue 104 until reaching the epidermis 101. Therefore, information (noise) on the oxygen saturation level of blood in blood vessels flowing through the dermis 102 and the subcutaneous tissue 103 is included. In contrast, the light detectors 12 arranged in the near area 14 receive a large amount of reflected light that has penetrated shallowly in the depth direction of the body 100. Therefore, the information on the oxygen saturation level (noise) of the blood in the blood vessels flowing through the dermis 102 and the subcutaneous tissue 103 can be obtained from the detection results of the light detectors 12 arranged in the near area 14. Thus, in order to calibrate the muscle oxygen saturation level (SmO₂) calculated from the detection results of the light detectors 12 arranged in the far area 15, the detection results of the light detectors 12 arranged in the near area 14 can be used, and the more accurate muscle oxygen saturation level (SmO₂) can be determined.

### Third Embodiment

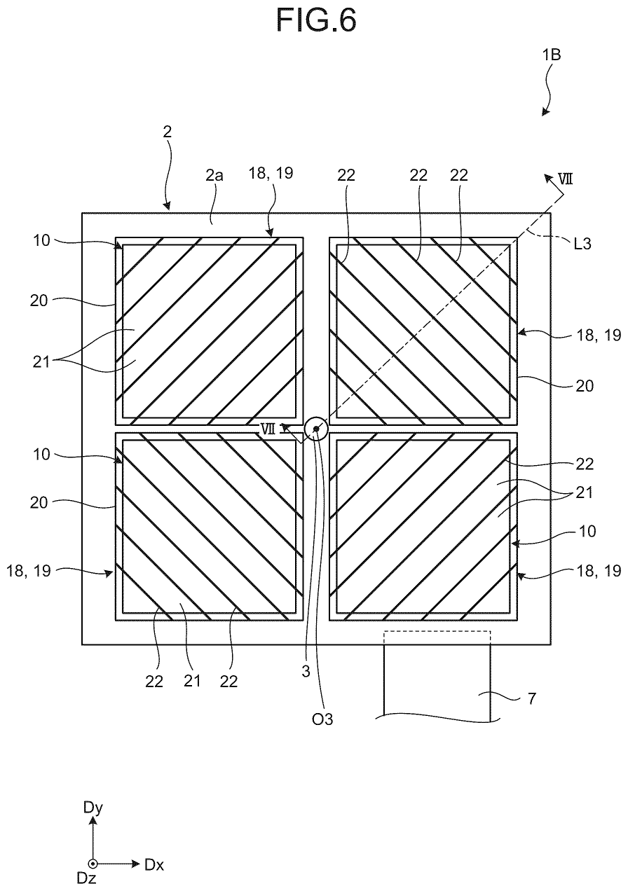

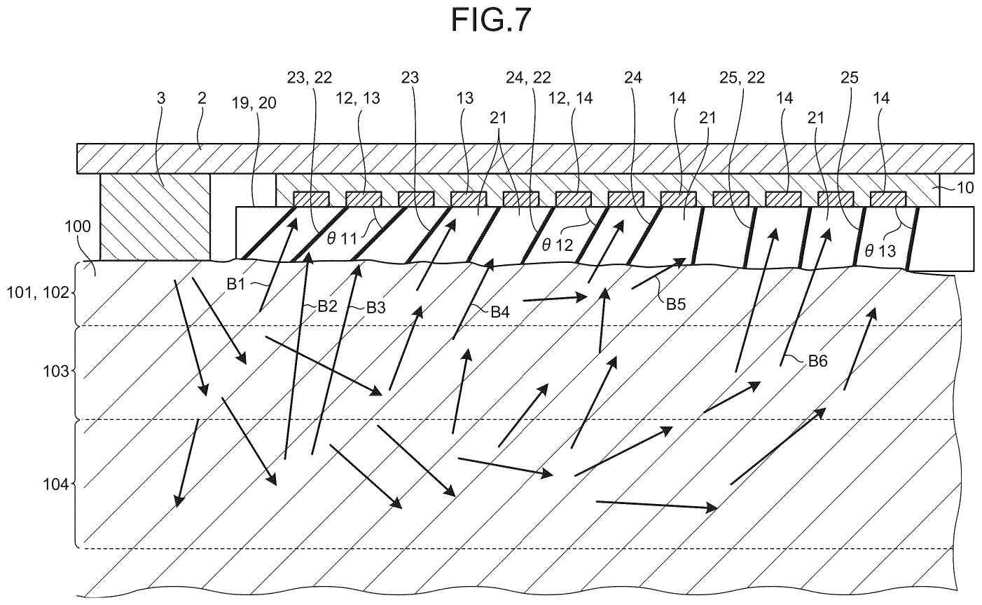

FIG. 6 is a plan view schematically illustrating a transcutaneous muscle oxygen saturation detection device according to a third embodiment of the present disclosure. FIG. 7 is a sectional view as viewed along arrows VII-VII in FIG. 6. As illustrated in FIG. 6, a transcutaneous muscle oxygen saturation detection device 1B of the third embodiment differs from the transcutaneous muscle oxygen saturation detection device 1A of the second embodiment in terms of including filters 18. The following describes the filters 18.

Each of the filters 18 is located between the light detection device 10 and the body 100 to define (limit) the angle of the reflected light incident on the light detectors 12. The filter 18 is provided for each of the light detection devices 10. The filter 18 of the third embodiment is a louver 19 provided with a plurality of first blades 22.

As illustrated in FIG. 7, the louver 19 is a resin layer 20 formed into a flat-plate shape and is fixed to a light-receiving surface of the light detection device 10. The light-receiving surface of the light detection device 10 is a surface on which the light detectors 12 are arranged and the reflected light is incident. The resin layer 20 includes a plurality of transmitting portions 21 and the first blades 22 that are black resin portions. The transmitting portions 21 are transparent resin portions through which light can pass. The first blades 22 are, in contrast, black resin portions and absorb light. The following describes details of the first blades 22.

The first blades 22 extend in the thickness direction of the resin layer 20 and are plate-shaped. As illustrated in FIG. 6, the first blades 22 extend orthogonally to an imaginary line L3 extending in a direction away from the light source 3 in plan view. The transmitting portions 21 and the first blades 22 are alternately arranged in the direction in which the imaginary line L3 extends. Each of the light detectors 12 is disposed so as to overlap a corresponding one of the transmitting portions 21. The imaginary line L3 intersects each of the first direction Dx and the second direction Dy at 45 degrees.

The first blades 22 include first inclined plates 23, second inclined plates 24, and third inclined plates 25 sequentially arranged from the side closest to the light source 3. In other words, the first blades 22 include the first inclined plates 23 arranged near the light source 3, the second inclined plates 24 arranged at locations farther than the first inclined plates 23 from the light source 3, and the third inclined plates 25 arranged at locations farther than the second inclined plates 24 from the light source 3. In the present embodiment, four each of the first inclined plates 23, the second inclined plates 24, and the third inclined plates 25 are provided.

The first inclined plates 23 overlap the near area 14. Therefore, the first inclined plates 23 limit the angle of the reflected light incident on the light detectors 12 arranged in the near area 14. Gaps between the second inclined plates 24 and gaps between the third inclined plates 25 overlap the far area 15. Therefore, the second inclined plates 24 and the third inclined plates 25 limit the angle of the reflected light incident on the light detectors 12 arranged in the far area 15.

As illustrated in FIG. 7, the first inclined plates 23, the second inclined plates 24, and the third inclined plates 25 are inclined so as to be closer to the light source 3 as being more away from the light-receiving surface. In detail, each of the first inclined plates 23 has an inclination angle θ11 of 50 degrees with respect to the light detection device 10. Each of the second inclined plates 24 has an inclination angle θ12 of 65 degrees with respect to the light detection device 10. Therefore, the inclination angle θ12 of the second inclined plates 24 is larger than the inclination angle θ11 of the first inclined plates 23. Each of the third inclined plates 25 has an inclination angle θ13 of 80 degrees with respect to the light detection device 10. As described above, the inclination angle of the first blades 22 with respect to the light detection device 10 increases as they are more apart from the light source 3.

The following describes advantages of the third embodiment. The reflected light emitted outside the body 100 near the light source 3 passes through the transmitting portions 21 between the first inclined plates 23 only when the reflected light is greatly inclined with respect to the normal direction of the light-receiving surface of the light detection device 10. Therefore, the reflected light that has penetrated shallowly in the depth direction of the body 100 (light reflected at the dermis 102 and the subcutaneous tissue 103 (refer to an arrow B1 in FIG. 7)) passes through the first inclined plates 23. In contrast, the reflected light that has penetrated deeply in the depth direction of the body 100 (light transmitted through the muscle tissue 104 (refer to arrows B2 and B3 in FIG. 7)) is not greatly inclined with respect to the normal direction of the light-receiving surface of the light detection device 10 and does not pass through the transmitting portions 21 between the first inclined plates 23. As described above, the light detectors 12 arranged in the near area 14 receives only the reflected light that has penetrated shallowly in the depth direction of the body 100. As a result, the accuracy of information on the oxygen saturation level (noise) of the dermis 102 and the subcutaneous tissue 103 calculated from the detection results of the light detectors 12 arranged in the near area 14 is improved.

In contrast, the reflected light emitted outside the body 100 far from the light source 3 passes through the transmitting portions 21 between the second inclined plates 24 or between the third inclined plates 25 only when the inclination angle of the reflected light is small with respect to the normal direction of the light-receiving surface of the light detection device 10. Therefore, the reflected light that has penetrated deeply in the depth direction of the body 100 (refer to arrows B4 and B6 in FIG. 7) passes through the gaps between the second inclined plates 24 or the gaps between the third inclined plates 25. In contrast, the reflected light that has penetrated shallowly in the depth direction of the body 100 (refer to an arrows B5 in FIG. 7) is greatly inclined with respect to the normal direction of the light-receiving surface of the light detection device 10 and does not pass through the transmitting portions 21 between the second inclined plates 24 and between the third inclined plates 25. Therefore, the light detectors 12 arranged in the far area 15 receive only the reflected light that has penetrated deeply in the depth direction of the body 100. As a result, the accuracy of the muscle oxygen saturation level (SmO₂) calculated from the detection results of the light detectors 12 arranged in the far area 15 is improved.

As described above, according to the third embodiment, the filter 18 (louver 19) separate the light into the light that has passed through the dermis 102 and the subcutaneous tissue 103 and the light that has passed through the muscle tissue 104, which are separately received by the light detectors 12. In other words, the resolution of each of the light detectors 12 is improved and the accurate muscle oxygen saturation level (SmO₂) can be detected.

The inclination angles θ11, θ12, and θ13 of the first, the second, and the third inclined plates 23, 24, and 25, respectively, in the third embodiment are exemplary, and may be different from the angles illustrated in the embodiment. The first blades 22 of the third embodiment have three different inclination angles (first inclined plates 23, second inclined plates 24, and third inclined plate 25), but may have two, or four or more inclination angles, and not limited to any number of inclination angles.

### Fourth Embodiment

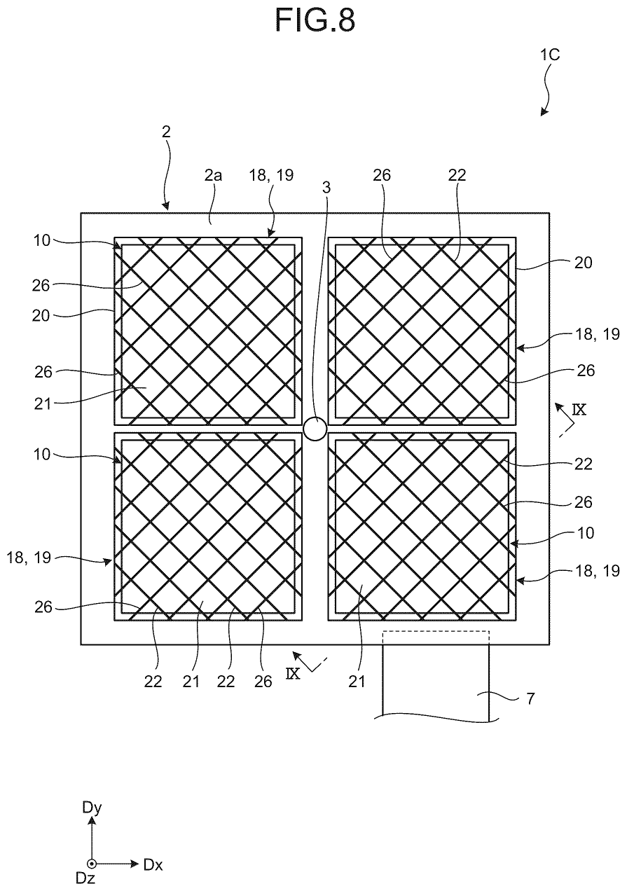

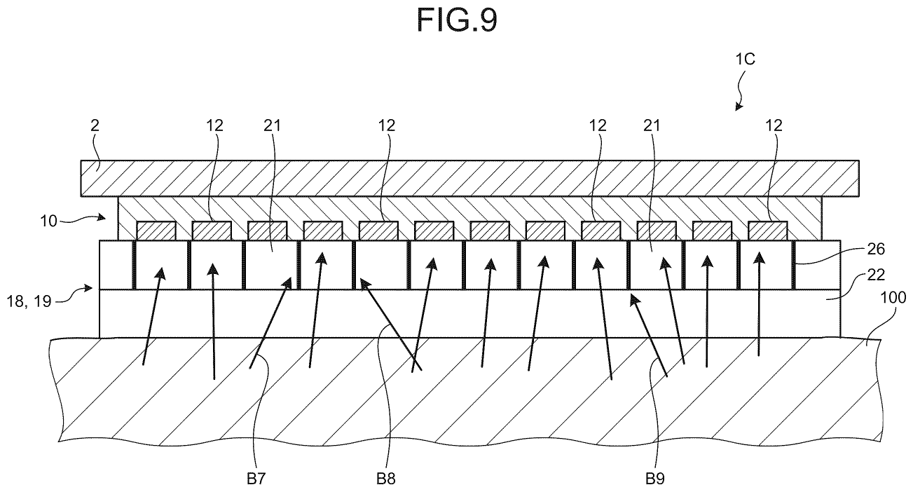

FIG. 8 is a plan view schematically illustrating a transcutaneous muscle oxygen saturation detection device according to a fourth embodiment of the present disclosure. FIG. 9 is a sectional view as viewed along arrows IX-IX in FIG. 8. As illustrated in FIG. 8, a transcutaneous muscle oxygen saturation detection device 1C of the fourth embodiment differs from the transcutaneous muscle oxygen saturation detection device 1B of the third embodiment in that the louver 19 of the transcutaneous muscle oxygen saturation detection device 1C includes a plurality of second blades 26. The following describes the second blades 26.

The second blades 26 are portions of the resin layer 20 and are plate-shaped black light-absorbing portions extending in the thickness direction. That is, in the same manner as the first blades 22, the second blades 26 have black outer surfaces and absorb the reflected light incident on the outer surfaces. As illustrated in FIG. 8, the second blades 26 extend in a direction orthogonal to the first blades 22. The second blades 26 are arranged at equal intervals in the direction in which the first blades 22 extend. The transmitting portions 21 are arranged between the second blades 26. As illustrated in FIG. 9, the second blades 26 are interposed between the first blades 22 and the light detection device 10. The transmitting portions 21 located between the second blades 26 are arranged so as to overlap the light detectors 12. The second blades 26 are inclined at an angle of 90 degrees with respect to the light detection device 10.

As described above, with the transcutaneous muscle oxygen saturation detection device 1C of the fourth embodiment, the reflected light emitted outside the body 100 passes through gaps between the first blades 22, and then passes through gaps between the second blades 26 and enters the light detectors 12. Of the reflected light emitted outside the body 100, the reflected light that is greatly inclined in the direction in which the first blades 22 extend (refer to arrows B7, B8, and B9 in FIG. 9) contacts the second blades 26 and does not enter the light detectors 12. As a result, each of the light detectors 12 receives only the reflected light from a corresponding area among the areas of the muscle tissue 104 divided in the first direction and the second direction. Thus, the resolution of each of the light detectors 12 is improved and the accurate muscle oxygen saturation level (SmO₂) can be detected.

### Fifth Embodiment

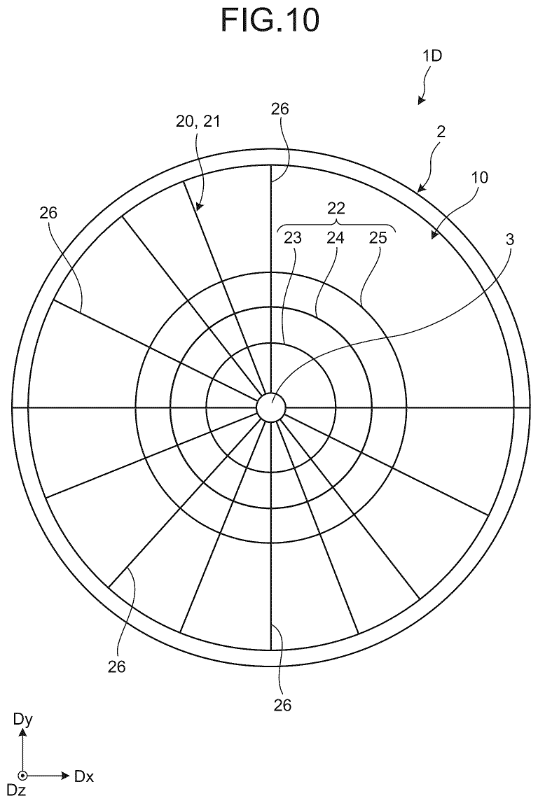

FIG. 10 is a plan view schematically illustrating a transcutaneous muscle oxygen saturation detection device according to a fifth embodiment of the present disclosure. As illustrated in FIG. 10, a transcutaneous muscle oxygen saturation detection device 1D of the fifth embodiment differs from the other embodiments in that each component has a circular shape centered on the light source 3.

In detail, each of the sheet 2 and the light detection device 10 has a circular shape centered on the light source 3. The light source 3 is fixed to a central portion of the light detection device 10. Each of the first blades 22 of the louver 19 has an annular shape centered on the light source 3. Three first blades 22 are provided. The three first blades 22 are, sequentially from the inner circumference, the first inclined plate 23, the second inclined plate 24, and the third inclined plate 25 that have different inclination angles. The second blades 26 radially extend around the light source 3. The light detectors (not illustrated) of the light detection device 10 are provided one in each of areas divided by the first blades 22 (first inclined plate 23, second inclined plate 24, and third inclined plate 25) and the second blades 26.

The same advantages as those of fourth embodiment can be obtained with the transcutaneous muscle oxygen saturation detection device 1D of the fifth embodiment. In the light detection device of the present disclosure, a plurality of light detectors may be arranged in each of the areas divided by the first blades 22 and the second blades 26.

As described above, in the first embodiment to the fifth embodiment, the transcutaneous muscle oxygen saturation detection devices 1, 1A, 1B, 1C, and 1D each including only one light source 3 have been described. However, the transcutaneous muscle oxygen saturation detection device of the present disclosure may include two or more light sources. The following describes a transcutaneous muscle oxygen saturation detection device including a plurality of light sources.

### Sixth Embodiment

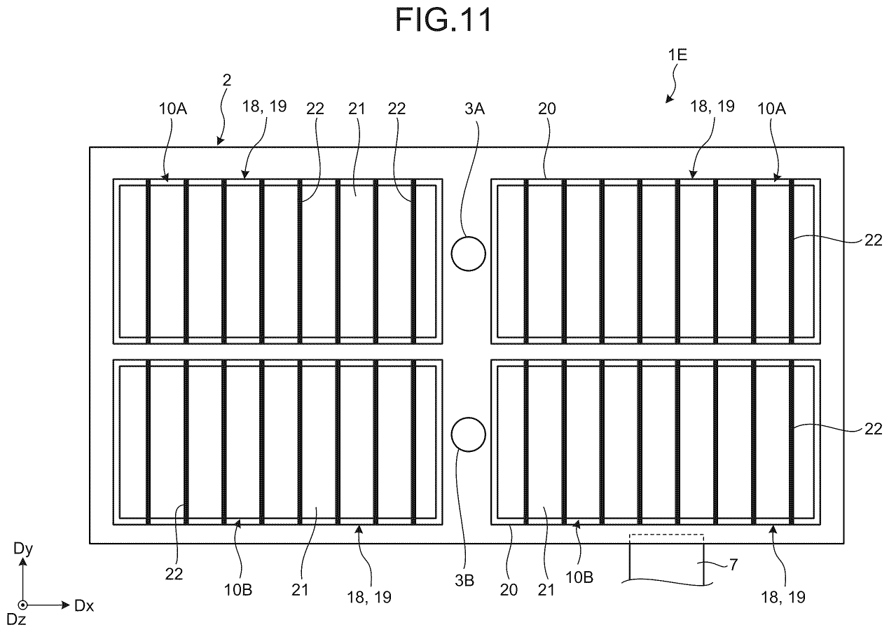

FIG. 11 is a plan view schematically illustrating a transcutaneous muscle oxygen saturation detection device according to a sixth embodiment of the present disclosure. A transcutaneous muscle oxygen saturation detection device 1E of the sixth embodiment includes one sheet 2, two light sources 3A and 3B, four light detection devices 10A and 10B, and four louvers 19.

The light sources 3A and 3B are fixed to a central portion in the first direction Dx of the sheet 2. The light sources 3A and 3B are separated from each other in the second direction Dy. Two light detection devices 10A are fixed to the sheet 2 so as to sandwich the light source 3A from both sides in the first direction Dx. Two light detection devices 10B are fixed to the sheet 2 so as to sandwich the light source 3B from both sides in the first direction Dx.

The louver 19 is provided for each of the light detection devices 10A and 10B. Each of the first blades 22 extends in the second direction Dy and the third direction Dz. The first blades 22 are arranged in the first direction Dx. The inclination angle of the first blade 22 with respect to the light detection device 10A or 10B increases (toward an angle orthogonal to the light detection device 10A or 10B) as the distance away from the light sources 3A and 3B increases.

As described above, with the transcutaneous muscle oxygen saturation detection device 1E of the sixth embodiment, the two light detection devices 10A receive light (reflected light) emitted from the light source 3A. The light detection devices 10B receive light (reflected light) emitted from the light source 3B. As a result, the muscle oxygen saturation level (SmO₂) in a wider area can be determined. Since the louvers 19 are provided, the reflected light that has penetrated shallowly in the depth direction of the body 100 enters the light detectors near the light sources 3A and 3B, and the reflected light that has penetrated deeply in the depth direction of the body 100 enters the light detectors far from the light sources 3A and 3B. Therefore, the resolution of the light detectors is high. The two light sources 3A and 3B may be turned on either simultaneously or sequentially.

### Seventh Embodiment

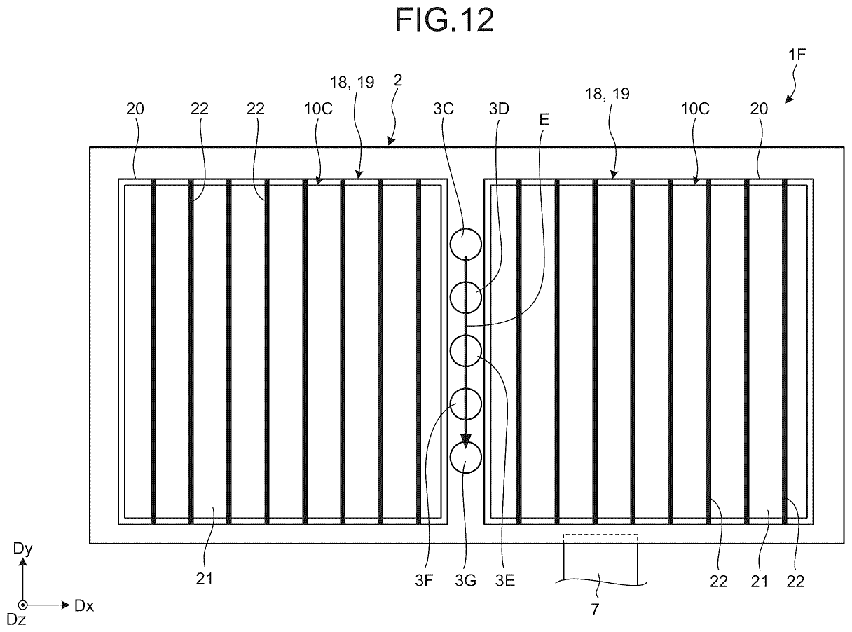

FIG. 12 is a plan view schematically illustrating a transcutaneous muscle oxygen saturation detection device according to a seventh embodiment of the present disclosure. As illustrated in FIG. 12, a transcutaneous muscle oxygen saturation detection device 1F of the seventh embodiment includes one sheet 2, five light sources 3C, 3D, 3E, 3F, and 3G, two light detection devices 10C, and two louvers 19.

The light sources 3C, 3D, 3E, 3F, and 3G are fixed to the central portion in the first direction Dx of the sheet 2. The light sources 3C, 3D, 3E, 3F, and 3G are arranged in the second direction Dy while being separated from one another. The two light detection devices 10C are arranged so as to sandwich the light sources 3C, 3D, 3E, 3F, and 3G from both sides in the first direction Dx. The inclination angle of the first blade 22 of the louver 19 with respect to the light detection device 10C increases as the distance away from the light sources 3C, 3D, 3E, 3F, and 3G increases.

The transcutaneous muscle oxygen saturation detection device 1F of the seventh embodiment turns on the light sources 3C, 3D, 3E, 3F, and 3G in this order as indicated by an arrow E in FIG. 12. The light detection devices 10C on both sides sequentially receive light (reflected light) emitted from the light sources 3C, 3D, 3E, 3F, and 3G. Therefore, the light detection devices 10C are shared by the light sources. As described above, the transcutaneous muscle oxygen saturation detection device 1F of the seventh embodiment can also determine the muscle oxygen saturation level (SmO₂) in a wider area. The louvers 19 are also provided and the resolution of the light detectors is high.

### Eighth Embodiment

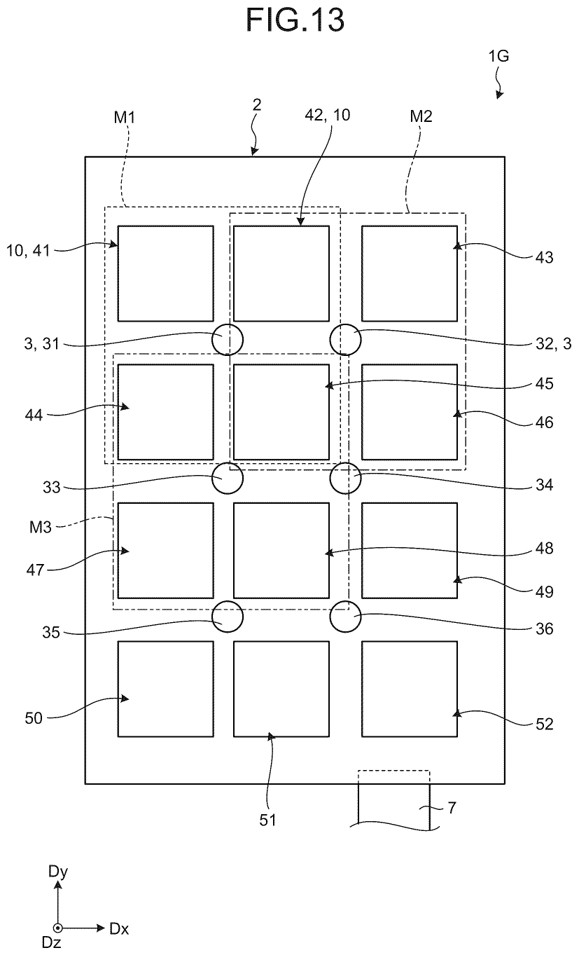

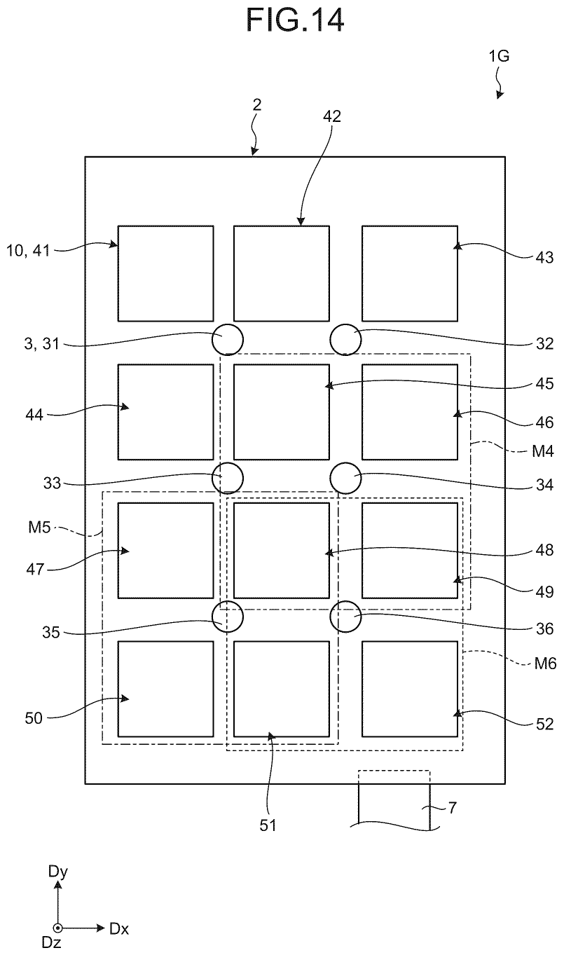

FIG. 13 is a plan view schematically illustrating a transcutaneous muscle oxygen saturation detection device according to an eighth embodiment, further illustrating areas of the light detection devices that are driven at first to third lighting times. FIG. 14 is a plan view illustrating areas of the light detection devices that are driven at fourth to sixth lighting times in the transcutaneous muscle oxygen saturation detection device of the eighth embodiment. As illustrated in FIG. 13, a transcutaneous muscle oxygen saturation detection device 1G of the eighth embodiment includes a sheet 2, six light sources 3, and twelve light detection devices 10.

The six light sources 3 (31, 32, 33, 34, 35, and 36) are arranged in a matrix having a row-column configuration, with two columns in the first direction Dx and three rows in the second direction Dy. The twelve light detection devices 10 (41, 42, 43, 44, 45, 46, 47, 48, 49, 50, 51, and 52) are arranged in a matrix having a row-column configuration, with three columns in the first direction Dx and four rows in the second direction Dy. Each of the light sources 3 is located at a central portion among the four light detection devices 10. Therefore, when one of the light sources 3 emits light, four of the light detection devices 10 receive the reflected light.

In the transcutaneous muscle oxygen saturation detection device 1G described above, the light sources 3 are turned on in the following order: the first light source 31, the second light source 32, the third light source 33, the fourth light source 34, the fifth light source 35, and the sixth light source 36. Therefore, at the first lighting time of the first light source 31, the light detection devices 41, 42, 44, and 45 surrounded by a dashed line M1 in FIG. 13 receive light (reflected light). At the next second lighting time of the second light source 32, the light detection devices 42, 43, 45, and 46 surrounded by a dashed line M2 in FIG. 13 receive light (reflected light). At the next third lighting time of the third light source 33, the light detection devices 44, 45, 47, and 48 surrounded by a dashed line M3 in FIG. 13 receive light (reflected light).

At the next fourth lighting time of the fourth light source 34, the light detection devices 45, 46, 48, and 49 surrounded by a dashed line M4 in FIG. 14 receive light (reflected light). At the next fifth lighting time of the fifth light source 35, the light detection devices 47, 48, 50, and 51 surrounded by a dashed line M5 in FIG. 14 receive light (reflected light). At the next sixth lighting time of the sixth light source 36, the light detection devices 48, 49, 51, and 52 surrounded by a dashed line M6 in FIG. 14 receive light (reflected light).

As described above, with the transcutaneous muscle oxygen saturation detection device 1G of the eighth embodiment, the muscle oxygen saturation level (SmO₂) in a wide area can be determined. In addition, the light detection devices 10 are shared, allowing the number of the light detection devices 10 to decrease.

While the first embodiment to the eighth embodiment have been described above, the transcutaneous muscle oxygen saturation detection device of the present disclosure is not limited to the examples described in the embodiments. For example, regarding the transcutaneous muscle oxygen saturation detection device 1C of the fourth embodiment, the radial second blades 26 described in the fifth embodiment may be provided instead of the second blades 26 orthogonal to the first blades 22. Although the louver 19 of the embodiments is made of the resin layer 20, the louver of the present disclosure may be made of material other than resin. Alternatively, the louver 19 may be provided on the transcutaneous muscle oxygen saturation detection device 1 of the first embodiment. While the example has been given where the louver 19 is used as the filter 18, a combination with pinholes or microlenses may be used as the filter. The oxygen saturation detection device of the present disclosure may be attached to the forehead other than the muscle tissue of an arm, a leg, or the like, and used, for example, to monitor activity of brain cells by measuring a change over time of the oxygen saturation level in the frontal lobe.

What is claimed is:

1. A transcutaneous muscle oxygen saturation detection device comprising:
a light source configured to emit light into a body; and
a first light detector and a second light detector that are configured to detect reflected light reflected in the body, wherein
the second light detector is circumferentially displaced about the light source from a first imaginary line connecting the light source to the first light detector.

2. The transcutaneous muscle oxygen saturation detection device according to claim 1, wherein the light source is configured to emit red light and infrared red.

3. The transcutaneous muscle oxygen saturation detection device according to claim 1, wherein a second imaginary line connecting the light source to the second light detector intersects the first imaginary line at a crossing angle of at least 5 degrees.

4. The transcutaneous muscle oxygen saturation detection device according to claim 1, comprising a light detection device provided with a plurality of light detectors on a substrate, wherein
the light detectors comprise the first light detector and the second light detector.

5. The transcutaneous muscle oxygen saturation detection device according to claim 4, comprising a plurality of the light detection devices.

6. The transcutaneous muscle oxygen saturation detection device according to claim 5, comprising a plurality of the light sources, wherein
the light detection devices are configured to sequentially detect reflected light of light emitted by the light sources.

7. The transcutaneous muscle oxygen saturation detection device according to claim 4, comprising a filter that is located between the light detection device and the body and is configured to define an angle of the reflected light emitted from inside the body and incident on the light detectors.

8. The transcutaneous muscle oxygen saturation detection device according to claim 7, wherein
the filter comprises a louver,
the light detection device has a light-receiving surface on which the reflected light is incident,
the louver comprises a plurality of first blades that divide the light-receiving surface in a direction away from the light source,
the first blades comprise first inclined plates arranged near the light source and second inclined plates that are arranged at locations more away from the light source than the first inclined plates are,
the first inclined plates and the second inclined plates are inclined so as to be closer to the light source as being more away from the light-receiving surface, and
the first inclined plates are inclined at a larger angle than that of the second inclined plates.

9. The transcutaneous muscle oxygen saturation detection device according to claim 8, wherein the louver comprises a plurality of second blades extending in a direction intersecting the first blades.
